(19) United States
(12) Patent Application Publication
(10) Pub. No.: US 2025/0084490 A1
(43) Pub. Date: Mar. 13, 2025

(54) METHOD FOR SIMULTANEOUS DETECTION AND QUANTIFICATION OF Listeria monocytogenes, Salmonella spp., AND SHIGA TOXIN-PRODUCING Escherichia coli (STEC)

(71) Applicants: UNIVERSIDAD DE CHILE, Santiago (CL); FUNDACION COPEC UNIVERSIDAD CATÓLICA, Santiago (CL)

(72) Inventors: Angélica REYES, Santiago (CL); Paola NAVARRETE, Santiago (CL); Gaétan CHINNICCI, Santiago (CL); Angel PARRA, Santiago (CL)

(21) Appl. No.: 18/563,954

(22) PCT Filed: May 26, 2021
(86) PCT No.: PCT/IB2021/054614

§ 371 Date: Nov. 24, 2023

(51) Int. Cl. C12Q 1/689; C12Q 1/6851

(52) U.S. Cl. CPC C12Q 1/689; C12Q 1/6851; C12Q 2600/166

(57) ABSTRACT

The invention refers to a method for simultaneous detection and quantification of Listeria monocytogenes, Salmonella spp. and Shiga toxin-producing Escherichia coli (STEC), from any kind of sample related to food production, including complex food matrices such as fish, meat, or fruit; or simple matrices such as water, or food contact surfaces. The method of invention allows specific quantification of the above-mentioned pathogens simultaneously thanks to specificity of designed primers for the qPCR reaction. It is also highly reliable because the method includes a system to adequately control the quantification of pathogens within the matrix. This system comprises the inoculation of samples with a known concentration of a transformed microorganism (host) carrying a chimeric sequence, acting as an internal control host for the whole process.

### TECHNICAL FIELD OF THE INVENTION

This invention refers to a method for simultaneous detection and quantification of Listeria monocytogenes, Salmonella spp. and Shiga toxin-producing Escherichia coli (STEC), from any kind of sample related with food production, including complex food matrices such as fish, meat, or fruit; or simple matrices such as water, or food contact surfaces.

### BACKGROUND INFORMATION

Bacterial foodborne diseases produced by pathogenic bacteria or through their toxins are one of the main alimentary risks in food products, such as fruit, meat, or fish.

Among the most dangerous bacteria, due to the severity of their related diseases, are L. monocytogenes, Salmonella spp. and Shiga toxin-producing Escherichia coli (STEC). For instance, after ingestion of contaminated food with STEC, the bacteria releases toxins known as Shiga toxins. These Shiga toxins provoke acute diarrhea, hemorrhagic colitis, and in some cases, acute kidney damage. Salmonella infections provoke vomiting, nausea and diarrhea. Listeriosis is a serious disease with symptoms ranging from febrile gastroenteritis to a severe invasive disease, and it especially affects pregnant women, children under 2 years, elders and immunocompromised people.

Therefore, food and sanitary regulations are strict to control and identify pathogens in food matrices. At the same time, since these microorganisms can survive within food processing, it is necessary to have exhaustive control of production processes, watching for possible contamination in food contact surfaces or utensils, in order to guarantee the safety of the final product.

Bacterial culture is the traditional method to identify and quantify foodborne pathogens in food. This method is based on identification of morphological, biochemical, physiological and immunological features from isolated bacterial colonies. However, even though this approach has been quite useful for many years, it has limitations such as being laborious and taking too long to obtain results. For example, the identification of any pathogen by culture method can take at least 4 days.

Time reduction for obtaining results thus becomes a critical aspect which directly impacts economics of agri-food industries. For example, it is commonly required to identify pathogens present in perishable food, so any delays in confirming food safety have important repercussions on storage costs while also reducing sales chances. Moreover, from a production process control perspective, rapid detection of possible contamination allows corrective actions to be taken on time, while also reducing possible cross contamination.

Furthermore, from a public health point of view, in the case of an outbreak; rapid identification also enables the control of sales of contaminated food (recall) and adequate treatment of affected people.

Rapidity has been accomplished through different molecular biology techniques, such as PCR, multiplex PCR and multiplex real-time PCR. Even though there are different PCR detection kits for these pathogens on the market, they are qualitative, in other words; they only screen for presence or absence of foodborne pathogens. The quantification of microorganisms present on a matrix at industrial level is relevant in order to evaluate sanitizing and safety protocols in the production process. However, there are no rapid quantitative methods available on the market. Additionally, it is still difficult to work with samples from food matrices using molecular biology tools, due to their complexity and the presence of inhibitors from the food which may affect or interfere with the results, giving false negatives. This situation offers an opportunity for developing rapid, simultaneous and quantitative methods for foodborne pathogens, which is the application field of this invention.

Given the importance of foodborne pathogen detection and quantification, there are several disclosed methods directed at screening those microorganisms, however methods known from the prior art don't have a system that ensures the effectiveness of the complete process, or only focus on one particular kind of complex matrix to be tested, while it remains unclear if said methods will work on other types of samples. In the following paragraphs, the closest prior art will be discussed:

Patent ES2540158 (B1) refers to a simultaneous screening method based on multiplex PCR for seven different pathogens, including E. coli 0157: H7, L. monocytogenes and Salmonella spp. as targeted sequences. An internal control corresponding to chimeric DNA is exclusively used in the amplification stage. The present invention differs to this patent, in the PCR primers used, the internal control (not only used to evaluate the amplification stage) as well as in additional steps, which will be described later.

Scientific publication from Wei C. et al. (Simultaneous detection of Escherichia coli 0157: H7, Staphylococcus aureus and Salmonella by multiplex PCR in milk. 3 Biotech, January 2018, 8:76) points out simultaneous detection (non-quantitative) from milk through multiplex PCR. Also, in this publication, internal control is used only in the amplification stage.

International PCT publication WO2016164407 (A2) refers to simultaneous detection of pathogens including STEC, L. monocytogenes and Salmonella spp., through qPCR. An internal DNA control is used in the amplification stage, requiring a microorganism enrichment step of 24 hours at 37° C. with an enrichment media prior to detection. Evidently, said enrichment step is performed to decrease false negative results, while artificially increasing pathogen concentration. Although this step makes pathogen detection easier; it makes impossible to determine original pathogen concentration of the actual food sample.

In conclusion, even where the prior art does provide simultaneous detection methods for the above mentioned three pathogens; there is still a need for a quantitative method which can be adaptable to different matrices, irrespective of its nature, like food, water or surfaces, and also allowing for a control of all the stages of the process in order to minimize the chances of obtaining false negatives. The internal control described has been designed to be included in the last part of the protocol to control the qPCR amplification reaction. However, they did not control the recovery of pathogens from the matrix, and the DNA extraction procedure.

The inventors have solved this technical issue with a new method including a control bacterium, which is added to the food sample to be analyzed before separating microorganisms from the matrix, and allowing for a control of all steps of the process; thus, decreasing the number of false negative results and allowing quantitative results to be obtained.

### BRIEF DESCRIPTION OF FIGURES

FIG. 1. Calibration curves for the quantification of the pathogens: a) L. monocytogenes, b) Salmonella spp. and c) STEC. All curves show Cq values versus each microorganism's concentration on samples (Log10 CFU/g or Log10 CFU/cm²).

### DETAILED DESCRIPTION OF THE INVENTION

The invention refers to a method for simultaneous detection and quantification of Listeria monocytogenes, Salmonella spp. and Shiga toxin-producing Escherichia coli (STEC), from any kind of sample related to food production, including complex food matrices such as fish, meat, or fruit; or simple matrices such as water, or food contact surfaces.

The invention allows specific quantification of the above-mentioned pathogens simultaneously thanks to specificity of designed primers for the qPCR reaction. It is also highly reliable because the method includes a system to adequately control the quantification of pathogens within the matrix. This system comprises the inoculation of samples with a known concentration of a transformed microorganism (host) carrying a chimeric sequence, acting as an internal control host for the whole process.

This way it is possible to maintain a precise control for the entire process and determine whether the result is valid or not. As previously established in the background section, the critical problem (unresolved until today) in determining the presence of these bacteria using molecular techniques, is the possibility for obtaining false negatives. Most methods for identifying these bacteria using PCR only control the last step of the process, which is the amplification of DNA (qPCR). There is lack of control in the first stages including separating bacteria from the sample and extraction of bacteria DNA. On the contrary, the present invention includes a unique control for all the stages which ensures pathogen collection from the matrix, DNA extraction and the qPCR reaction.

Accordingly, the invention consists in a method for simultaneous detection and quantification of Listeria monocytogenes, Salmonella spp. and Shiga toxin-producing Escherichia coli (STEC) from different matrices, including a host (like a bacterium) as internal control (I.C.) which ensures correct application of all process steps. It is important to note that the invention can be applied to detect and quantify 1 or simultaneously 2 or 3 of pathogens using the same internal control host. This invention then comprises the following steps:
(a) adding a known quantity of internal control host (I.C.) to the sample, wherein the internal control host carrying a polynucleotide comprising a chimeric polynucleotide sequence;
(b) assessing the sample for the detection and quantification of nucleic acids of internal control (I.C.) and L. monocytogenes, Salmonella spp., Shiga toxin-producing Escherichia coli (STEC), or combinations thereof;
(c) determining the pathogen concentration by standard curve analysis made for each of the pathogens;
(d) validating the process with the results for I. C. which must give a signal according to the concentration used in (a).

Regarding the I.C., a person skilled in the art should consider evident that selecting a microorganism species to be transformed does not represent a critical issue, thus any bacterial, archaea or yeast species available in the prior art can be used. However, the transformed bacterium species preferably used is an Escherichia coli, which does not produce Shiga toxins and is non-pathogenic.

Similarly, the nature of the chimeric sequence also does not affect the method of detection and quantification of the pathogens, while the sequence included would be artificial and not-homologous to any bacterial genomes under study. Conveniently for the method of invention, the inventors used a chimeric sequence constructed with a region with the sequence of one of the primers of the invention, separated by a region of 20 to 70 bp from a third region with the sequence of another of the primers of the invention. This way, the chimeric sequence incorporated in the control host carries a sequence of a forward primer selected from SEQ ID No. 1, 3, 5, 7, 9, 11, 13, 15, and 17; a 20 to 70 bp central-linker region; and a complementary sequence to a reverse primer selected from SEQ ID No 2, 4, 6, 8, 10, 12, 14, 16 and 18. Conveniently both primers are chosen from two different pathogens. In a preferred embodiment of the invention, the central linker region have 26 bp, and its sequence is that defined in SEQ ID No. 19.

So, in a first embodiment, said chimeric polynucleotide sequence is selected from the combinations of any of SEQ ID No 1, 3, 5 with any complementary of SEQ ID No 8, 10, 12, 14, 16, 18 (i.e. SEQ ID No39, 40, 41, 42, 43 and 44), wherein the central-linker region is SEQ ID No 19, i.e. SEQ ID No 21, 45 to 61.

In a second embodiment, said chimeric polynucleotide sequence is selected from the combinations of any of SEQ ID No 7, 9, 11 with any complementary of SEQ ID No 2, 4, 6, 14, 16, 18 (i.e. SEQ ID No 36, 37, 38, 42, 43 and 44), wherein the central-linker region is SEQ ID No 19, i.e. SEQ ID No 62 to 79.

In a third embodiment said chimeric polynucleotide sequence is selected from the combinations of any of SEQ ID No 13, 15, 17 with any complementary of SEQ ID No 2, 4, 6, 8, 10, 12 (i.e. SEQ ID No 36, 37, 38, 39, 40 and 41), wherein the central-linker region is SEQ ID No 19, i.e. SEQ ID No 20, 80 to 96.

Preferred examples of chimeric sequences that can be used according to this invention are SEQ ID No. 20 and 21. For instance, SEQ ID No. 20 is formed by a region which is equivalent to Salmonella spp., the primer defined in SEQ ID No. 13, a 26 bp region sequence as defined in SEQ ID No. 19, and a region complementary to STEC, the primer defined in SEQ ID No. 8 (i.e. SEQ ID No 38). And SEQ ID No. 21 is formed by a region which is equivalent to Listeria monocytogenes, the primer defined in SEQ ID No. 1, a 26 bp region sequence as defined in SEQ ID No. 19, and a region complementary to STEC, the primer defined in SEQ ID No. 10 (i.e. SEQ ID No 40).

The method of invention further comprises a step to separate microorganisms out of the sample. Wherein said step of microorganism separation is performed by filtration, centrifugation, sorting, magnetic beads or combinations thereof.

The method of invention further comprises a step of nucleic acids extraction from the sample.

The method of invention wherein said nucleic acids detection is performed by PCR, RT-PCR, qPCR, digital PCR, LinDA, DNA microarray, PCR coupled with high-throughput sequencing, PCR DGGE/TTGE, or combinations thereof.

Wherein said nucleic acids detection of L. monocytogenes is performed using primers comprising a sequence selected from SEQ ID No. 1 to 6, its derivatives, or combinations thereof; and when said nucleic acids detection of L. monocytogenes is performed by qPCR use probes comprising a sequence selected from SEQ ID No. 22 to 25, its derivatives, or combinations thereof.

Wherein said nucleic acids detection of STEC is performed using primers comprising a sequence selected from SEQ ID No. 7 to 12, its derivatives, or combinations thereof; and when said nucleic acids detection of STEC is performed by qPCR, use probes comprising a sequence selected from SEQ ID No. 26 to 29, its derivatives, or combinations thereof.

Wherein said nucleic acids detection of Salmonella spp. bacteria is performed using primers comprising a sequence selected from SEQ ID No. 13 to 18, its derivatives, or combinations thereof and when said nucleic acids of Salmonella spp. bacteria is performed by qPCR use probes comprising a sequence selected from SEQ ID No. 30 to 33, its derivatives, or combinations thereof.

Wherein said nucleic acids detection of IC is performed using as forward primer a sequence selected from SEQ ID No. 1, 3, 5, 7, 9, 11, 13, 15, and 17, its derivatives, or combinations thereof; and as reverse primer a sequence selected from SEQ ID No 2, 4, 6, 8, 10, 12, 14, 16, and 18, its derivatives, or combinations thereof; and when said nucleic acids detection of IC is performed by qPCR use probes comprising a sequence selected from SEQ ID No. 34 to 35, its derivatives, or combinations thereof.

In the method of the invention (steps (a) to (d)), said (c) step comprises a step of determining Listeria monocytogenes, Salmonella spp. bacteria and or Shiga toxin-producing Escherichia coli (STEC) concentration in a sample, interpolating the Cq value obtained by qPCR obtained in step (b) on a standard curve for each pathogen Listeria monocytogenes, Salmonella spp. bacteria and/or Shiga toxin producing Escherichia coli (STEC) concentration and validating it by the value for I.C. signal obtained in step (d).

The method of invention allows work with different types of samples, like biological sample selected from meat, poultry, seafood, sausages, dairy, fruits, vegetables, ready to eat foods, beverages, or water, or surfaces.

The I.C. host is added to the sample at a concentration of 10²-10¹⁰ cells or CFU per mL.

The polynucleotide to obtain an IC useful to detect and quantify Listeria monocytogenes, Salmonella spp., bacteria and/or Shiga toxin-producing Escherichia coli (STEC) in a sample, wherein said polynucleotide comprises:
a sequence of a forward primer selected from SEQ ID No. 1, 3, 5, 7, 9, 11, 13, 15 and 17; a 26 bp central-linker region; and a complementary sequence to a reverse primer selected from SEQ ID No 2, 4, 6, 8, 10, 12, 14, 16 and 18 (i.e. SEQ ID No. 36 to 44). Wherein said central-linker region in a preferred embodiment is SEQ ID No. 19.

In a first embodiment, the polynucleotide is selected from the combinations of any of SEQ ID No 1, 3, 5 with any complementary of SEQ ID No 8, 10, 12, 14, 16, 18 (i.e. SEQ ID No39, 40, 41, 42, 43 and 44), wherein the central-linker region is SEQ ID No 19, i.e. SEQ ID No 21, 45 to 61.

In a second embodiment, the polynucleotide is selected from the combinations of any of SEQ ID No 7, 9, 11 with any complementary of SEQ ID No 2, 4, 6, 14, 16, 18 (i.e. SEQ ID No 36, 37, 38, 42, 43 and 44), wherein the central-linker region is SEQ ID No 19, i.e. SEQ ID No 62 to 79.

In a third embodiment, the polynucleotide is selected from the combinations of any of SEQ ID No 13, 15, 17 with any complementary of SEQ ID No 2, 4, 6, 8, 10, 12 (i.e. SEQ ID No 36, 37, 38, 39, 40 and 41), wherein the central-linker region is SEQ ID No 19, i.e. SEQ ID No 20, 80 to 96.

In a particularly preferred embodiment said polynucleotide chimeric comprises a sequence selected from a sequence selected from SEQ ID No 20 (formed by SEQ ID No 13, 19 and 8) or SEQ ID No. 21 (formed by SEQ ID No 1, 19 and 10).

The polynucleotide can be included in plasmid, cassette, episome, DNA construct, or combinations thereof, and is carried by a host selected from bacteria, archaea or yeast. Wherein said host is, transformed, edited or transfected with said polynucleotide. In one preferred embodiment said host is Escherichia coli.

In one preferred embodiment of the invention, E. coli K12 (host) is used for the construction of the internal control, then transformed with vector pGEM®-T Easy (Vector Systems-Promega Corporation) comprising a chimeric sequence as defined previously, and the transformants are selected using 100 μg/mL ampicillin.

As previously established, the sample to be assessed is inoculated with a previously known quantity of the internal control host corresponding to a bacterium (E. coli K-12) transformed with a vector carrying chimeric sequence. Preferably, adding a 10-200 μl aliquot of a bacterial suspension of 10²-10¹⁰ CFU/mL.

As stated above, the internal control host (I.C.) is a bacterium transformed with the chimeric sequence, for the person skilled in the art it will be evident that there is a linear correlation between the concentration of the transformed bacteria and the chimeric sequence. So, depending on the stage of the process that is discussed, the term I.C. is used in this text to refer interchangeably to both terms, transformed control bacteria host or chimeric sequence.

The step referring to the microorganism's separation from the sample can be performed using any prior art methods depending on the nature of the sample. In one embodiment of the invention, separating all microorganisms from the sample is performed, for instance, from water only by filtration; from food by detachment washes using detergent solutions and then filtration; from surfaces by surface swabbing, resuspension in transport media and further filtration; and so on.

After microorganisms are collected, the DNA extraction step proceeds, which may be accomplished using any techniques available in the prior art. Then, DNA is resuspended in nuclease-free water.

Quantitative PCR (qPCR) for L. monocytogenes, Salmonella spp., STEC and the chimeric sequence is performed using DNA extracted from the samples. The qPCR result is validated in accordance with the Cq value for the chimeric sequence. In cases where the result is not consistent with the expected value, the measurement must be repeated.

In order to determine sample pathogen concentrations, it should be evident for a person skilled in this area, to construct a calibration curve or standard curve based on concentration standards known for each one of the pathogens to be assessed, versus the amplification cycle (Ct or Cq) related to qPCR technique. This way, the method of this invention provides Ct or Cq for each pathogen (L. monocytogenes, Salmonella spp. and STEC). Therefore, the concentration of each microorganism in the studied sample is determined by interpolating the obtained Ct value in the qPCR reaction in the standard curve for each pathogen.

Since the concentration of inoculated control bacteria host is previously known as well as their corresponding expected Cq value, the method can be validated by simply using this Cq value.

The following preferred embodiments of the invention are described merely as examples, without limiting any technical variants that a person skilled in the art may incorporate or modify, and which are therefore also included within the scope of the inventive concept claimed in this document.

### EXAMPLES

### Example 1. Quantification of Listeria monocytogenes, Salmonella Spp. and Shiga Toxin-Producing Escherichia coli (STEC) in Salmon and a Comparison with the Traditional Method

To establish the advantage of using the method of this invention, target pathogens L. monocytogenes, Salmonella spp. and STEC were determined in parallel by using the method of the present invention as well as by the traditional “gold standard” culture method, applied to samples inoculated with different quantities of these pathogens.

The traditional method consists of seeding the sample in different culture plates containing specific media for each bacterium for 24 to 48 hours and counting the colony-forming units (CFU), then performing identification tests such as conventional PCR for each microorganism.

### 1.1 Inoculation of Pathogens Using Known Concentrations and Addition of I.C. (Internal Control Host).

Ten raw fresh salmon samples, 100 g each, were inoculated with different concentrations of the 3 pathogens as described in Table 1, 2 and 3. Samples were processed with the method of this invention and the gold standard test (culture technique). Samples processed using the method of this invention were also inoculated with 25 μl of I.C. at a concentration of 10² CFU/mL.

I.C. was obtained through transformation of E. coli K12 with vector pGEM®-T Easy (Vector Systems—Promega Corporation) which carries a chimeric sequence defined as SEQ ID No. 31 designed with the reaction primers. To inoculate samples with I.C., this transformed bacterium was grown in LB (Luria-Bertani) broth plus 100 μg/mL ampicillin.

### 1.2 Conventional Method (Gold Standard)

Quantification of L. monocytogenes was performed using the protocol described in the Bacteriological Analytical Manual (Hitchins, A., Jinneman, K., and Chen, Y. Chapter 10. Detection of Listeria monocytogenes in Foods and Environmental Samples, and Enumeration of Listeria monocytogenes in Foods, available on line). The quantification of Salmonella spp. was performed using the method described in: Brichta-Harhay D M, Arthur T M, Koohmaraie M, Enumeration of Salmonella from poultry carcass rinses via direct plating methods. Lett Appl Microbiol. 2008; 46 (2): 186-191. doi: 10.1111/j.1472-765X.2007.02289.x. There is no gold standard method for STEC quantification.

### 1.3 Method of the Invention

For separation of bacteria from the matrix a detergent solution was added to samples. Samples resuspended in detergent solution were filtered using a sterile gauze, and then the filtrate was passed through a 0.45 μm pore diameter nitrocellulose filter, to retain bacteria. Filtration was performed using a vacuum pump.

The Nitrocellulose filter, containing the bacteria, was recovered using sterile tweezers and then put in a 2 mL centrifuge tube in order to proceed with DNA sample extraction.

Then, bacteria DNA extraction was performed through standard extraction technology using phenol: chloroform. This protocol allows lysing bacteria through enzymatic (lysozyme and proteinase) and chemical action (sodium dodecyl sulfate or SDS). Then the DNA is finally resuspended in nuclease-free water or a TE (Tris-EDTA) buffer.

Once the DNA is obtained, qPCR is performed for detection of each pathogen and the chimeric sequence. For this 1 μl of resuspended DNA was used for each reaction, in triplicate.

Primers SEQ ID No. 1 and 2 were used for L. monocytogenes; primers SEQ ID No. 9 and 10 for STEC; primers SEQ ID No. 15 and 16 for Salmonella spp. and primers SEQ ID No. 1 and 10 for the chimeric sequence (I.C.). The probes used for detection of L. monocytogenes (SEQ ID No. 24), Salmonella spp. (SEQ ID No. 33), STEC (SEQ ID No. 26) and IC (SEQ ID No. 34).

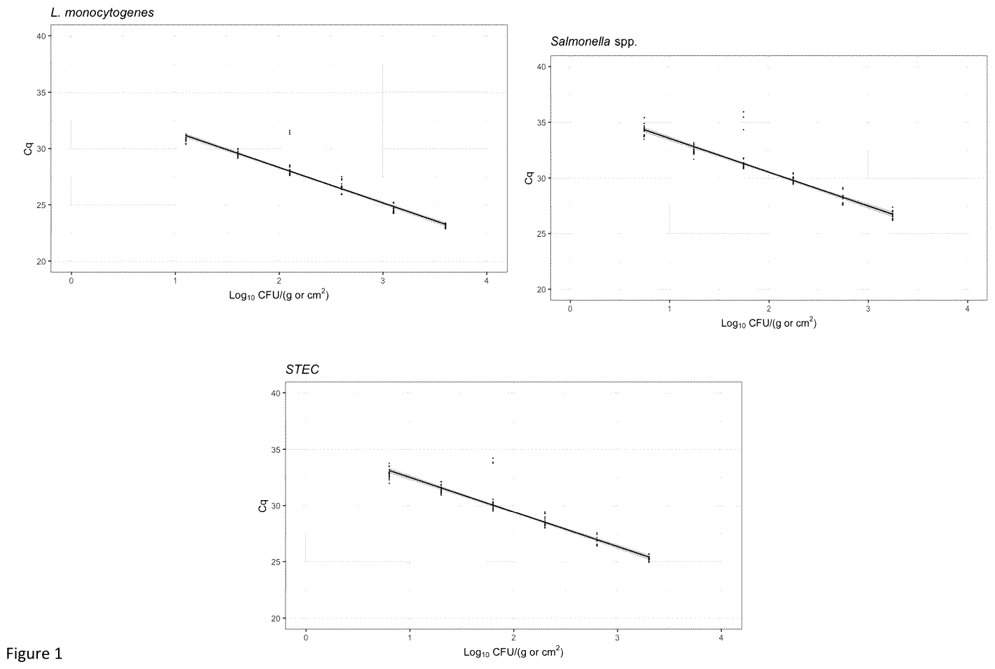

After the qPCR reaction, a Cq value was obtained for each pathogen and the I.C. The concentration of each pathogen was estimated in CFU/g of salmon using a standard curve previously constructed (Cq v/s CFU/g, as shown in FIG. 1). Lower Cq means a higher concentration of each pathogen expressed as CFU/g.

The results obtained using the method of the present invention are shown in Table 1 for L. monocytogenes, Table 2 for Salmonella spp. and Table 3 for STEC. The tables include the concentration of each microorganism added (Inoculum) to each sample, and the results obtained applying a Gold standard test. In addition, tables show the Cq value obtained for the I.C. In order to validate the assay, the Cq value for the IC must be in a 28 to 33.5 range.

TABLE 1
Comparative results for quantification of L. monocytogenes
from salmon using the method of the present invention
and the Gold standard test.

| Sample | Log₁₀ CFU/g | Log₁₀ CFU/g | Log₁₀ CFU/g | Cq |
|---|---|---|---|---|
| ID | Inoculum | Gold Standard | Invention | Internal Control |
| S1 | NI* | <0.6** | <1.59** | 29.2 |
| S2 | NI* | <0.6** | <1.59** | 29.5 |
| S3 | NI* | <0.6** | <1.59** | 30.9 |
| S4 | 2.1 | <0.6** | <1.59** | 30.4 |
| S5 | 2.2 | 2.0 | 2.02 | 29.5 |
| S6 | 2.3 | 2.5 | 1.85 | 29.4 |
| S7 | 3.0 | 1.9 | 2.48 | 29.2 |
| S8 | 3.2 | <0.6** | 3.06 | 30.4 |
| S9 | 3.2 | 2.7 | 3.00 | 30.6 |
| S10 | 3.3 | 3.5 | 2.88 | 29.7 |

NI*non-inoculated.
**Detection limit of each method.

TABLE 2
Comparative results for quantification of Salmonella
spp. from salmon using the method of the present
invention and the Gold standard test.

| Sample | Log₁₀ CFU/g | Log₁₀ CFU/g | Log₁₀ CFU/g | Cq |
|---|---|---|---|---|
| ID | Inoculum | Gold Standard | Invention | Internal Control |
| S1 | NI* | <0.6** | <1.76** | 29.2 |
| S2 | NI* | <0.6** | <1.76** | 29.5 |
| S3 | NI* | <0.6** | <1.76** | 30.9 |
| S4 | 0.5 | <0.6** | <1.76** | 29.8 |
| S5 | 0.9 | <0.6** | <1.76** | 30.4 |
| S6 | 2.47 | 1.4 | 1.76 | 29.5 |
| S7 | 2.54 | 1.4 | 2.05 | 29.2 |
| S8 | 3.48 | 2.3 | 2.52 | 30.6 |
| S9 | 3.60 | 1.4 | 2.69 | 30.4 |
| S10 | 3.88 | 2.3 | 3.28 | 29.7 |

NI*non-inoculated.
**Detection limit of each method.

TABLE 3
Comparative results for quantification of STEC from salmon using
the method of the present invention. There is no Gold Standard
test for quantifying this pathogen from salmon samples.

| Sample | Log₁₀ CFU/g | Log₁₀ CFU/g | Log₁₀ CFU/g | Cq |
|---|---|---|---|---|
| ID | Inoculum | Gold Standard | Invention | Internal Control |
| S1 | NI* | NA** | <1.64*** | 29.2 |
| S2 | NI* | NA** | <1.64*** | 29.5 |
| S3 | NI* | NA** | <1.64*** | 30.9 |
| S4 | 0 | NA** | <1.64*** | 29.8 |
| S5 | 0.2 | NA** | <1.64*** | 30.4 |
| S6 | 2.0 | NA** | 1.72 | 29.2 |
| S7 | 2.6 | NA** | <1.64*** | 29.5 |
| S8 | 3.2 | NA** | 2.71 | 29.7 |
| S9 | 3.4 | NA** | 2.91 | 30.4 |
| S10 | 3.6 | NA** | 2.48 | 30.6 |

NI*non-inoculated;
**NA: gold standard quantification method is not available;
***Detection limit

When comparing the results obtained for L. monocytogenes, Salmonella spp., and STEC using the method of this invention, they show similar quantification limits (Log101.59, Log101.76, and Log101.64 CFU/g respectively). The method of the present invention showed a ten-fold higher quantification limit compared to the limit of the gold standard method. For the STEC quantification, there is no standard protocol available to compare with our results and we compared our results with the inoculum added to the samples.

The results indicate that, when applying the method of this invention, most of the concentration values obtained are closer to the inoculum added to the sample, than those obtained through the gold standard method.

Considering the time taken since the sample processing initiated, the method of this invention took only 8 hours, while for the traditional test 2 days were needed to get results. Accordingly, the method of the invention delivers similar results to those obtained using gold standard tests in a substantially shorter period of time.

### Example 2. Quantification of Listeria monocytogenes, Salmonella Spp. and Shiga Toxin-Producing Escherichia coli (STEC) in the Transport Media Used for the Surface Testing System

There are different sampling protocols for the microbiological analyses of surfaces, which are standardized. Among these protocols, there is sample collection through a sterile swab that is repeatedly rubbed (in different directions) over a 100 cm² area. Afterwards, this swab is submerged in Letheen transport media.

### 2.1 Inoculation of Pathogens Using Known Concentrations and Addition of I.C. (Internal Control Host).

In order to contrast results using the method of this invention with the gold standard test, swabs submerged in Letheen buffer (20 mL) were inoculated with different pathogen concentrations to be analyzed as detailed in Tables 4, 5 and 6. Afterwards, 10 ml of inoculated Letheen buffer was processed using the method of the present invention, while another 10 mL was analyzed through the gold standard method. Samples processed using the method of this invention were also inoculated with 25 μL of I.C. at a 10² CFU/ml concentration.

### 2.2 Gold Standard Method.

Similarly, as explained in example 1.2, quantification of pathogens L. monocytogenes, and Salmonella spp. present in surface samples were processed by the protocols described in the Bacteriological Analytical Manual (gold standard tests) and by Brichta-Harhay, et al (2008).

There is no gold standard method for STEC quantification, so quantification of this pathogen was determined only using the method of the present invention.

### 2.3 Method of the Invention

Separation of bacteria from media: The Letheen broth was filtrated through a 0.45 μm pore diameter nitrocellulose filter, which is capable of retaining these bacteria. Filtration was performed using a vacuum pump.

The filter, containing the bacteria, was recovered using sterile tweezers and then put in a 2 mL centrifuge tube.

Then, DNA extraction of the bacteria present in the filter was performed through standard extraction technology using phenol: chloroform, in accordance with the details of the previous example. The DNA was resuspended in nuclease-free water or a TE (for Tris-EDTA) buffer.

Once the DNA is obtained, qPCR is performed for detection of each pathogen and the chimeric sequence. 1 μL of suspended DNA was used for each reaction, in triplicate.

Primers SEQ ID No. 3 and 4 were used for L. monocytogenes; primers SEQ ID No. 7 and 8 for STEC; primers SEQ ID No. 13 and 14 for Salmonella spp. and primers SEQ ID No. 13 and 8 for the chimeric sequence (I.C.). The probes used for detection of L. monocytogenes (SEQ ID No. 25), Salmonella spp. (SEQ ID No. 32), STEC (SEQ ID No. 27) and IC (SEQ ID No. 35).

After the qPCR reaction, a Cq value was obtained for each pathogen and the I.C. The concentration of each pathogen was estimated in CFU/cm² of the surface using a standard curve previously constructed (Cq v/s CFU/cm², as shown in FIG. 1).

The results after using the method of the invention are shown in Table 4 for L. monocytogenes, in Table 5 for Salmonella spp. and in Table 6 for STEC. The tables include the concentration of each microorganism added (Inoculum) to each sample, and the results obtained applying a Gold standard test. In addition, the tables show the Cq value obtained for the I.C. in order to validate the assay, the Cq value for the IC must be in a 28 to 33.5 range.

TABLE 4
Comparative quantification results of L. monocytogenes
from surface samples using the method of the present
invention and the Gold standard test.

| | | | Log₁₀ | Cq |
|---|---|---|---|---|
| Sample | Log₁₀ CFU/cm² | Log₁₀ CFU/cm² | bacterium/cm² | Internal |
| ID | Inoculum | Gold Standard | Invention | Control |
| S1 | NI | <0** | <0.98** | 28.55 |
| S2 | NI | <0** | <0.98** | 28.39 |
| S3 | NI | <0** | <0.98** | 28.66 |
| S4 | 0.95 | 2.51 | <0.98** | 32.33 |
| S5 | 0.95 | 2.54 | <0.98** | 28.64 |
| S6 | 0.95 | 1.18 | <0.98** | 28.52 |
| S7 | 1.95 | 2.20 | 2.27 | 32.08 |
| S8 | 1.95 | 2.08 | 2.28 | 28.20 |
| S9 | 1.95 | 3.48 | 2.25 | 28.54 |
| S10 | 2.95 | 3.00 | 3.35 | 28.57 |
| S11 | 2.95 | 3.73 | 3.27 | 33.52 |
| S12 | 2.95 | 3.69 | 3.13 | 28.92 |

NI*non-inoculated.
**Detection limit (corresponds to 1 CFU/cm² for the Gold Standard method)

TABLE 5
Comparative quantification results of Salmonella
spp. from surface samples using the method of the
present invention and the Gold standard test.

| | | | Log₁₀ | Cq |
|---|---|---|---|---|
| Sample | Log₁₀ CFU/cm² | Log₁₀ CFU/cm² | bacterium/cm² | Internal |
| ID | Inoculum | Gold Standard | Invention | Control |
| S1 | NI* | <0** | <1.16** | 28.55 |
| S2 | NI* | <0** | <1.16** | 28.39 |
| S3 | NI* | <0** | <1.16** | 28.66 |
| S4 | 0.60 | 2.75 | <1.16** | 32.33 |
| S5 | 0.60 | 3.51 | <1.16** | 28.64 |
| S6 | 0.60 | 1.26 | <1.16** | 28.52 |
| S7 | 1.60 | 2.28 | 1.16 | 32.08 |
| S8 | 1.60 | 1.72 | 1.18 | 28.20 |
| S9 | 1.60 | 3.71 | <1.16** | 28.54 |
| S10 | 2.60 | 2.83 | 2.04 | 28.57 |
| S11 | 2.60 | 4.00 | 1.99 | 33.52 |
| S12 | 2.60 | 4.04 | 2.00 | 28.92 |

NI*non-inoculated.
**Detection limit (corresponds to 1 CFU/cm² for the Gold Standard method)

TABLE 6
Comparative quantification results of STEC from surface samples using
the method of the present invention and the Gold standard test.

| | | | Log₁₀ | Cq |
|---|---|---|---|---|
| Sample | Log₁₀ CFU/cm² | Log₁₀ CFU/cm² | bacterium/cm² | Internal |
| ID | Inoculum | Gold Standard | Invention | Control |
| S1 | NI* | NA** | <1.04*** | 28.55 |
| S2 | NI* | NA** | <1.04*** | 28.39 |
| S3 | NI* | NA** | <1.04*** | 28.66 |
| S4 | 0.48 | NA** | <1.04*** | 32.33 |
| S5 | 0.48 | NA** | <1.04*** | 28.64 |
| S6 | 0.48 | NA** | <1.04*** | 28.52 |
| S7 | 1.48 | NA** | 2.17 | 32.08 |
| S8 | 1.48 | NA** | 2.24 | 28.20 |
| S9 | 1.48 | NA** | 2.09 | 28.54 |
| S10 | 2.48 | NA** | 3.11 | 28.57 |
| S11 | 2.48 | NA** | 3.08 | 33.52 |
| S12 | 2.48 | NA** | 3.05 | 28.92 |

NI*non-inoculated.
NA**gold standard quantification method is not available;
***Detection limit

Results show that it is possible to quantify L. monocytogenes, Salmonella spp., and STEC using the method of the invention at concentrations higher than Log100.98, Log101.16 and Log101.04 CFU/cm² respectively, that corresponds to the detection limit of the method for this matrix.

Additionally, values determined by the method of this invention are closer to the concentration inoculated to the surface sample, compared with those quantified by the gold standard test.

Internal control was successfully detected using the method of this invention for all samples, as expected, and the Cq values for this IC fluctuated between 28 and 33.5 as predicted for this I.C.

On the other hand, it is important to highlight that using the method of the present invention the results were obtained 8 hours after the start of the sample processing, compared to more than 2 days required for the gold standard method. Moreover, the method of the present invention quantifies three pathogens simultaneously while the gold standard processes each one separately.

1. A method for simultaneous or independent detection and quantification of Listeria monocytogenes, Salmonella spp. and Shiga toxin-producing Escherichia coli (STEC) in a sample comprising the following steps:
(e) adding a known quantity of internal control host (I.C.) to the sample, wherein the internal control host carrying a polynucleotide comprising a chimeric polynucleotide sequence;
(f) assessing the sample for the detection and quantification of nucleic acids of internal control (I.C.) and L. monocytogenes, Salmonella spp., Shiga toxin-producing Escherichia coli (STEC), or combinations thereof;
(g) determining the pathogen concentration by standard curve analysis made for each of the pathogens;
(h) validating the process with the results for I. C. which must give a signal according to the concentration used in (a).

2. The method according to claim 1, wherein the internal control host is selected from bacteria, archaea or yeast.

3. The method of claim 2, wherein the said host is Escherichia coli.

4. The method of claim 1, wherein said chimeric polynucleotide sequence comprises: a sequence of a forward primer selected from SEQ ID No. 1, 3, 5, 7, 9, 11, 13, 15, and 17; a 20 to 70 bp central-linker region; and a complementary sequence to a reverse primer selected from SEQ ID No 2, 4, 6, 8, 10, 12, 14, 16 and 18.

5. The method according to claim 4 wherein the complementary reverse sequence is selected from SEQ ID No. 36 to 44.

6. The method according to claim 4, wherein said central-linker region is SEQ ID No. 19.

7. The method according to claim 4, wherein said chimeric polynucleotide sequence is selected from the combinations of any of SEQ ID No 1, 3, 5 with any complementary of SEQ ID No 8, 10, 12, 14, 16, 18, wherein the central-linker region is SEQ ID No 19.

8. The method according to claim 7, wherein the full length sequence of said chimeric polynucleotide sequence is selected from SEQ ID No 21, 45 to 61.

9. The method according to claim 4, wherein said chimeric polynucleotide sequence is selected from the combinations of any of SEQ ID No 7, 9, 11 with any complementary of SEQ ID No 2, 4, 6, 14, 16, 18, wherein the central-linker region is SEQ ID No 19.

10. The method according to claim 9, wherein the full length sequence of said chimeric polynucleotide sequence is selected from SEQ ID No 62-79.

11. The method according to claim 4, wherein said chimeric polynucleotide sequence is selected from the combinations of any of SEQ ID No 13, 15, 17 with any complementary of SEQ ID No 2, 4, 6, 8, 10, 12, wherein the central-linker region is SEQ ID No 19.

12. The method according to claim 11, wherein the full length sequence of said chimeric polynucleotide sequence is selected from SEQ ID No 20, 80-96.

13. The method of claim 2, wherein said host is a transformed, edited or transfected host carrying the polynucleotide comprising the chimeric sequence selected from SEQ ID No. 20, 21, 44 to 96.

14. The method of claim 1, wherein said method further comprises a step to separate microorganisms out of the sample.

15. The method of claim 14, wherein said step of microorganism separation is performed by filtration, centrifugation, sorting, magnetic beads or combinations thereof.

16. The method of claim 15, wherein said method further comprises a step of nucleic acids extraction from the sample.

17. The method of claim 1, wherein said nucleic acids detection is performed by PCR, RT-PCR, qPCR, digital PCR, LinDA, DNA microarray, PCR coupled with high-throughput sequencing, PCR DGGE/TTGE, or combinations thereof.

18. The method of claim 17, wherein for said nucleic acids detection of L. monocytogenes is performed using primers comprising a sequence selected from SEQ ID No. 1 to 6, its derivatives, or combinations thereof.

19. The method of claim 18, wherein said nucleic acids detection of L. monocytogenes is performed by qPCR using probes comprising a sequence selected from SEQ ID No. 22 to 25, its derivatives, or combinations thereof.

20. The method of claim 17 wherein for said nucleic acids detection of STEC is performed using primers comprising a sequence selected from SEQ ID No. 7 to 12, its derivatives, or combinations thereof.

21. The method of claim 20, wherein said nucleic acids detection of STEC is performed by qPCR using probes comprising a sequence selected from SEQ ID No. 26 to 29, its derivatives, or combinations thereof.

22. The method of claim 17, wherein for said nucleic acids detection of Salmonella-type bacteria is performed using primers comprising a sequence selected from SEQ ID No. 13 to 18, its derivatives, or combinations thereof.

23. The method of claim 22, wherein said nucleic acids of Salmonella spp. bacteria is performed by qPCR using probes comprising a sequence selected from SEQ ID No. 30 to 33, its derivatives, or combinations thereof.

24. The method of claim 1, wherein said c) step comprises a step of determining Listeria monocytogenes, Salmonella spp. bacteria and or Shiga toxin-producing Escherichia coli (STEC) concentration in a sample interpolating the Cq value, obtained by qPCR in step b), on a standard curve for each pathogen Listeria monocytogenes, Salmonella spp. bacteria and/or Shiga toxin producing Escherichia coli (STEC) concentration and validating it by the value for I.C. signal obtained in step d).

25. The method of claim 24, wherein said nucleic acids detection of IC is performed using as forward primer a sequence selected from SEQ ID No. 1, 3, 5, 7, 9, 11, 13, 15, and 17, its derivatives, or combinations thereof; and as reverse primer a sequence selected from SEQ ID No 2, 4, 6, 8, 10, 12, 14, 16, and 18, its derivatives, or combinations thereof.

26. The method of claim 25, wherein said nucleic acids detection of IC is performed by qPCR using probes comprising a sequence selected from SEQ ID No. 34 to 35, its derivatives, or combinations thereof.

27. The method of claim 1, wherein said sample is a biological sample selected from meat, poultry, seafood, sausages, dairy, fruits, vegetables, ready to eat foods, beverages, water or surfaces.

28. The method of claim 1, wherein the I.C. host is added at a concentration of 10²-10¹⁰ cells or CFU per mL.

29. A polynucleotide to obtain an IC useful to detect and quantify Listeria monocytogenes, Salmonella spp., and/or Shiga toxin-producing Escherichia coli (STEC) in a sample, wherein said polynucleotide comprises:
a sequence of a forward primer selected from SEQ ID No. 1, 3, 5, 7, 9, 11, 13, 15 and 17;
a 26 bp central-linker region; and a complementary sequence to a reverse primer selected from SEQ ID No 2, 4, 6, 8, 10, 12, 14, 16 and 18.

30. The polynucleotide according to claim 29 wherein the complementary reverse sequence is selected from SEQ ID No. 36 to 44.

31. The polynucleotide of claim 29, wherein said central-linker region is SEQ ID No. 19.

32. The polynucleotide of claim 29 wherein said polynucleotide is selected from the combinations of any of SEQ ID No 1, 3, 5 with any complementary of SEQ ID No 8, 10, 12, 14, 16, 18, wherein the central-linker region is SEQ ID No 19.

33. The polynucleotide according to claim 32 wherein the full length sequence of said polynucleotide sequence is selected from SEQ ID No 21, 45 to 61.

34. The polynucleotide of claim 29, wherein said polynucleotide is selected from the combinations of any of SEQ ID No 7, 9, 11 with any complementary of SEQ ID No 2, 4, 6, 14, 16, 18, wherein the central-linker region is SEQ ID No 19.

35. The polynucleotide according to claim 34, wherein the full length sequence of said polynucleotide sequence is selected from SEQ ID No 62-79.

36. The polynucleotide of claim 29, wherein said polynucleotide is selected from the combinations of any of SEQ ID No 13, 15, 17 with any complementary of SEQ ID No 2, 4, 6, 8, 10, 12, wherein the central-linker region is SEQ ID No 19.

37. The polynucleotide according to claim 36, wherein the full length sequence of said polynucleotide sequence is selected from SEQ ID No 20, 80-96.

38. The polynucleotide according to claim 31 wherein said polynucleotide chimeric comprises a sequence selected from SEQ ID No 20, 21, 44 to 96.

39. The polynucleotide according to claim 31, wherein said polynucleotide is included in plasmid, cassette, episome, DNA construct, or combinations thereof.

40. The polynucleotide according to claim 29 wherein said polynucleotide is carried by a host selected from bacteria, archaea or yeast.

41. The polynucleotide of claim 40, wherein said host is, transformed, edited or transfected with said polynucleotide.

42. The polynucleotide according to claim 31 wherein said host is Escherichia coli.


SEQUENCE LISTING (96 sequences)

SEQ ID NO: 1 - DNA, 22 bp, Artificial Sequence
  Primer_bind (forward) to Listeria monocytogenes
aattcttccttcaaagccgtaa

SEQ ID NO: 2 - DNA, 21 bp, Artificial sequence
  primer_bind reverse Listeria monocytogenes
gaggttaccgtcgatgatttg

SEQ ID NO: 3 - DNA, 20 bp, Artificial sequence
  primer_bind forward Listeria monocytogenes
tgcgcaacaaactgaagcaa

SEQ ID NO: 4 - DNA, 20 bp, Artificial sequence
  primer_bind reverse Listeria monocytogenes
tggcgtcttaggacttgcag

SEQ ID NO: 5 - DNA, 23 bp, Artificial sequence
  primer_bind forward Listeria monocytogenes
tgcatctgcattcaataaagaaa

SEQ ID NO: 6 - DNA, 24 bp, Artificial sequence
  primer_bind reverse Listeria monocytogenes
tccgcgtgtttcttttcgattggc

SEQ ID NO: 7 - DNA, 23 bp, Artificial sequence
  primer_bind forward Shiga toxin-producing Escherichia coli (STEC)
ctatactccgattcctctggtga

SEQ ID NO: 8 - DNA, 23 bp, Artificial sequence
  primer_bind reverse Shiga toxin-producing Escherichia coli (STEC)
gatcattttcattacccgtacca

SEQ ID NO: 9 - DNA, 20 bp, Artificial sequence
  primer_bind forward Shiga toxin-producing Escherichia coli (STEC)
tttaggttcggcacctcttg

SEQ ID NO: 10 - DNA, 20 bp, Artificial sequence
  primer_bind reverse Shiga toxin-producing Escherichia coli (STEC)
ccttgtcatcggtcatgttg

SEQ ID NO: 11 - DNA, 20 bp, Artificial sequence
  primer_bind forward Shiga toxin-producing Escherichia coli (STEC)
caacatgaccgatgacaagg

SEQ ID NO: 12 - DNA, 20 bp, Artificial Sequence
  primer_bind reverse Shiga toxin-producing Escherichia coli (STEC)
gcggtatctttcgcgtaatc

SEQ ID NO: 13 - DNA, 21 bp, Artificial Sequence
  primer_bind forward Salmonella spp.
cggactcaccaggagattaca

SEQ ID NO: 14 - DNA, 19 bp, Artificial Sequence
  primer_bind reverse Salmonella spp.
ggcgagaccgacttttagc

SEQ ID NO: 15 - DNA, 20 bp, Artificial Sequence
  primer_bind forward Salmonella spp.
cttttagcgaagccatggag

SEQ ID NO: 16 - DNA, 20 bp, Artificial Sequence
  primer_bind reverse Salmonella spp.
gtttacccacgcgtttcatc

SEQ ID NO: 17 - DNA, 20 bp, Artificial Sequence
  primer_bind forward Salmonella spp.
cgctgtttaccggatttctc

SEQ ID NO: 18 - DNA, 20 bp, Artificial Sequence
  primer_bind reverse Salmonella spp.
cggccggtataaatcaacag

SEQ ID NO: 19 - DNA, 26 bp, Artificial sequence
  Center region to intenal control (IC)
acgtagtggcctcggcgccagtagct

SEQ ID NO: 20 - DNA, 70 bp, Artificial sequence
  Example of internal control, contructec with SEQ ID No 13, SEQ ID NO 19 and reverse of SEQ ID No 8 (i.e. SEQ ID No 39)
cggactcaccaggagattacaacgtagtggcctcggcgccagtagcttggtacgggtaatgaaaatgatc

SEQ ID NO: 21 - DNA, 68 bp, Artificial sequence
  Example of Intenal control constructed with SEQ ID No 1, SEQ ID No 9, and reverse complementary of SEQ ID No 10 (i.e. SEQ ID No 40)
aattcttccttcaaagccgtaaacgtagtggcctcggcgccagtagctcaacatgaccgatgacaagg

SEQ ID NO: 22 - DNA, 20 bp, Artificial Sequence
  Probe to Listeria monocytogenes
ccgccaagaaaaggttacaa

SEQ ID NO: 23 - DNA, 21 bp, Artificial Sequence
  Probe to Listeria monocytogenes
cggaggttccgcaaaagatga

SEQ ID NO: 24 - DNA, 21 bp, Artificial Sequence
  Probe to Listeria monocytogenes
atctgcattcaataaagaaaa

SEQ ID NO: 25 - DNA, 23 bp, Artificial Sequence
  Probe to Listeria monocytogenes
catccatggcaccaccagcatct

SEQ ID NO: 26 - DNA, 25 bp, Artificial Sequence
  Probe to Shiga toxin-producing Escherichia coli (STEC)
aggtggtgttgctggtcacacgaat

SEQ ID NO: 27 - DNA, 22 bp, Artificial Sequence
  Probe to Shiga toxin-producing Escherichia coli (STEC)
cgatggggatcgattaccgtca

SEQ ID NO: 28 - DNA, 24 bp, Artificial Sequence
  Probe to Shiga toxin-producing Escherichia coli (STEC)
taaattatgcggcacaacaggcgg

SEQ ID NO: 29 - DNA, 23 bp, Artificial Sequence
  Probe to Shiga toxin-producing Escherichia coli (STEC)
tgctggtcacacgaataaactga

SEQ ID NO: 30 - DNA, 27 bp, Artificial Sequence
  Probe to Salmonella spp.
catggctaatttaacccgtcgtcagtg

SEQ ID NO: 31 - DNA, 26 bp, Artificial Sequence
  Probe to Salmonella spp.
gtggttcctggcgcagcaaataaaac

SEQ ID NO: 32 - DNA, 27 bp, Artificial Sequence
  Probe to Salmonella spp.
gagaaagcggtcttgacgcccgctcaa

SEQ ID NO: 33 - DNA, 27 bp, Artificial Sequence
  Probe to Salmonella spp.
ctggcgcagcaaataaaacgattattc

SEQ ID NO: 34 - DNA, 21 bp, Artificial Sequence
  Probe to Internal control
cagtggcctcggcgccagtag

SEQ ID NO: 35 - DNA, 21 bp, Artificial Sequence
  Probe to Internal control
ccgtagtggcctcggcgccag

SEQ ID NO: 36 - DNA, 21 bp, Artificial Sequence
  Reverse complement  of primer_bind reverse 2 Listeria monocytogenes
caaatcatcgacggtaacctc

SEQ ID NO: 37 - DNA, 20 bp, Artificial Sequence
  Reverse complement  of primer_bind reverse 4 Listeria monocytogenes
ctgcaagtcctaagacgcca

SEQ ID NO: 38 - DNA, 24 bp, Artificial Sequence
  Reverse complement of primer_bind reverse 6 Listeria monocytogenes
gccaatcgaaaagaaacacgcgga

SEQ ID NO: 39 - DNA, 23 bp, Artificial Sequence
  Reverse complement primer_bind reverse 8 Shiga toxin-producing Escherichia coli (STEC)
tggtacgggtaatgaaaatgatc

SEQ ID NO: 40 - DNA, 20 bp, Artificial Sequence
  Reverse complement primer_bind reverse 10 Shiga toxin-producing Escherichia coli (STEC)
caacatgaccgatgacaagg

SEQ ID NO: 41 - DNA, 20 bp, Artificial Sequence
  Reverse complement primer_bind reverse 12 Shiga toxin-producing Escherichia coli (STEC)
gattacgcgaaagataccgc

SEQ ID NO: 42 - DNA, 19 bp, Artificial Sequence
  Reverse complement primer_bind reverse 14 Salmonella spp.
gctaaaagtcggtctcgcc

SEQ ID NO: 43 - DNA, 20 bp, Artificial Sequence
  Reverse complement primer_bind reverse 16 Salmonella spp.
gatgaaacgcgtgggtaaac

SEQ ID NO: 44 - DNA, 20 bp, Artificial Sequence
  Reverse complement primer_bind reverse 18 Salmonella spp.
ctgttgatttataccggccg

SEQ ID NO: 45 - DNA, 71 bp, Artificial Sequence
  Example of Intenal control constructed with SEQ ID No 1, SEQ ID No 19, and reverse complementary of SEQ ID No 8 (i.e. SEQ ID No 39)
aattcttccttcaaagccgtaaacgtagtggcctcggcgccagtagcttggtacgggtaatgaaaatgatc

SEQ ID NO: 46 - DNA, 68 bp, Artificial Sequence
  Example of Intenal control constructed with SEQ ID No 1, SEQ ID No 19, and reverse complementary of SEQ ID No 12 (i.e.SEQ ID No 41)
aattcttccttcaaagccgtaaacgtagtggcctcggcgccagtagctgattacgcgaaagataccgc

SEQ ID NO: 47 - DNA, 67 bp, Artificial Sequence
  Example of Intenal control constructed with SEQ ID No 1, SEQ ID No 19, and reverse complementary of SEQ ID No 14 (i.e. SEQ ID No 42)
aattcttccttcaaagccgtaaacgtagtggcctcggcgccagtagctgctaaaagtcggtctcgcc

SEQ ID NO: 48 - DNA, 68 bp, Artificial Sequence
  Example of Intenal control constructed with SEQ ID No 1, SEQ ID No 19, and reverse complementary of SEQ ID No 16 (i.e. SEQ ID No 43)
aattcttccttcaaagccgtaaacgtagtggcctcggcgccagtagctgatgaaacgcgtgggtaaac

SEQ ID NO: 49 - DNA, 68 bp, Artificial Sequence
  Example of Intenal control constructed with SEQ ID No 1, SEQ ID No 19, and reverse complementary of SEQ ID No 18 (i.e. SEQ ID No 44)
aattcttccttcaaagccgtaaacgtagtggcctcggcgccagtagctctgttgatttataccggccg

SEQ ID NO: 50 - DNA, 69 bp, Artificial Sequence
  Example of Intenal control constructed with SEQ ID No 3, SEQ ID No 19, and reverse complementary of SEQ ID No 8 (i.e. SEQ ID No 39)
tgcgcaacaaactgaagcaaacgtagtggcctcggcgccagtagcttggtacgggtaatgaaaatgatc

SEQ ID NO: 51 - DNA, 66 bp, Artificial Sequence
  Example of Intenal control constructed with SEQ ID No 3, SEQ ID No 19, and reverse complementary of SEQ ID No 10 (i.e. SEQ ID No 40)
tgcgcaacaaactgaagcaaacgtagtggcctcggcgccagtagctcaacatgaccgatgacaagg

SEQ ID NO: 52 - DNA, 66 bp, Artificial Sequence
  Example of Intenal control constructed with SEQ ID No 3, SEQ ID No 19, and reverse complementary of SEQ ID No 12 (i.e. SEQ ID No 41)
tgcgcaacaaactgaagcaaacgtagtggcctcggcgccagtagctgattacgcgaaagataccgc

SEQ ID NO: 53 - DNA, 65 bp, Artificial Sequence
  Example of Intenal control constructed with SEQ ID No 3, SEQ ID No 19, and reverse complementary of SEQ ID No 14 (i.e. SEQ ID No 42)
tgcgcaacaaactgaagcaaacgtagtggcctcggcgccagtagctgctaaaagtcggtctcgcc

SEQ ID NO: 54 - DNA, 66 bp, Artificial Sequence
  Example of Intenal control constructed with SEQ ID No 3, SEQ ID No 19, and reverse complementary of SEQ ID No 16 (i.e. SEQ ID No 43)
tgcgcaacaaactgaagcaaacgtagtggcctcggcgccagtagctgatgaaacgcgtgggtaaac

SEQ ID NO: 55 - DNA, 66 bp, Artificial Sequence
  Example of Intenal control constructed with SEQ ID No 3, SEQ ID No 19, and reverse complementary of SEQ ID No 18 (i.e. SEQ ID No 44)
tgcgcaacaaactgaagcaaacgtagtggcctcggcgccagtagctctgttgatttataccggccg

SEQ ID NO: 56 - DNA, 72 bp, Artificial Sequence
  Example of Intenal control constructed with SEQ ID No 5, SEQ ID No 19, and reverse complementary of SEQ ID No 8 (i.e. SEQ ID No 39)
tgcatctgcattcaataaagaaaacgtagtggcctcggcgccagtagcttggtacgggtaatgaaaatgatc

SEQ ID NO: 57 - DNA, 69 bp, Artificial Sequence
  Example of Intenal control constructed with SEQ ID No 5, SEQ ID No 19, and reverse complementary of SEQ ID No 10 (i.e. SEQ ID No 40)
tgcatctgcattcaataaagaaaacgtagtggcctcggcgccagtagctcaacatgaccgatgacaagg

SEQ ID NO: 58 - DNA, 69 bp, Artificial Sequence
  Example of Intenal control constructed with SEQ ID No 5, SEQ ID No 19, and reverse complementary of SEQ ID No 12 (i.e. SEQ ID No 41)
tgcatctgcattcaataaagaaaacgtagtggcctcggcgccagtagctgattacgcgaaagataccgc

SEQ ID NO: 59 - DNA, 68 bp, Artificial Sequence
  Example of Intenal control constructed with SEQ ID No 5, SEQ ID No 19, and reverse complementary of SEQ ID No 14 (i.e. SEQ ID No 42)
tgcatctgcattcaataaagaaaacgtagtggcctcggcgccagtagctgctaaaagtcggtctcgcc

SEQ ID NO: 60 - DNA, 69 bp, Artificial Sequence
  Example of Intenal control constructed with SEQ ID No 5, SEQ ID No 19, and reverse complementary of SEQ ID No 16 (i.e. SEQ ID No 43)
tgcatctgcattcaataaagaaaacgtagtggcctcggcgccagtagctgatgaaacgcgtgggtaaac

SEQ ID NO: 61 - DNA, 69 bp, Artificial Sequence
  Example of Intenal control constructed with SEQ ID No 5, SEQ ID No 19, and reverse complementary of SEQ ID No 18 (i.e. SEQ ID No 44)
tgcatctgcattcaataaagaaaacgtagtggcctcggcgccagtagctctgttgatttataccggccg

SEQ ID NO: 62 - DNA, 70 bp, Artificial Sequence
  Example of Intenal control constructed with SEQ ID No 7, SEQ ID No 19, and reverse complementary of SEQ ID No 2 (i.e. SEQ ID No 36)
ctatactccgattcctctggtgaacgtagtggcctcggcgccagtagctcaaatcatcgacggtaacctc

SEQ ID NO: 63 - DNA, 69 bp, Artificial Sequence
  Example of Intenal control constructed with SEQ ID No 7, SEQ ID No 19, and reverse complementary of SEQ ID No 4 (i.e. SEQ ID No 37)
ctatactccgattcctctggtgaacgtagtggcctcggcgccagtagctctgcaagtcctaagacgcca

SEQ ID NO: 64 - DNA, 73 bp, Artificial Sequence
  Example of Intenal control constructed with SEQ ID No 7, SEQ ID No 19, and reverse complementary of SEQ ID No 6 (i.e. SEQ ID No 38)
ctatactccgattcctctggtgaacgtagtggcctcggcgccagtagctgccaatcgaaaagaaacacgcgga

SEQ ID NO: 65 - DNA, 68 bp, Artificial Sequence
  Example of Intenal control constructed with SEQ ID No 7, SEQ ID No 19, and reverse complementary of SEQ ID No 14 (i.e. SEQ ID No 42)
ctatactccgattcctctggtgaacgtagtggcctcggcgccagtagctgctaaaagtcggtctcgcc

SEQ ID NO: 66 - DNA, 69 bp, Artificial Sequence
  Example of Intenal control constructed with SEQ ID No 7, SEQ ID No 19, and reverse complementary of SEQ ID No 16 (i.e. SEQ ID No 43)
ctatactccgattcctctggtgaacgtagtggcctcggcgccagtagctgatgaaacgcgtgggtaaac

SEQ ID NO: 67 - DNA, 69 bp, Artificial Sequence
  Example of Intenal control constructed with SEQ ID No 7, SEQ ID No 19, and reverse complementary of SEQ ID No 18 (i.e. SEQ ID No 44)
ctatactccgattcctctggtgaacgtagtggcctcggcgccagtagctctgttgatttataccggccg

SEQ ID NO: 68 - DNA, 67 bp, Artificial Sequence
  Example of Intenal control constructed with SEQ ID No9, SEQ ID No 19, and reverse complementary of SEQ ID No 2 (i.e. SEQ ID No 36)
tttaggttcggcacctcttgacgtagtggcctcggcgccagtagctcaaatcatcgacggtaacctc

SEQ ID NO: 69 - DNA, 66 bp, Artificial Sequence
  Example of Intenal control constructed with SEQ ID No 9, SEQ ID No 19, and reverse complementary of SEQ ID No 4 (i.e. SEQ ID No 37)
tttaggttcggcacctcttgacgtagtggcctcggcgccagtagctctgcaagtcctaagacgcca

SEQ ID NO: 70 - DNA, 70 bp, Artificial Sequence
  Example of Intenal control constructed with SEQ ID No 9, SEQ ID No 19, and reverse complementary of SEQ ID No 6 (i.e. SEQ ID No 38)
tttaggttcggcacctcttgacgtagtggcctcggcgccagtagctgccaatcgaaaagaaacacgcgga

SEQ ID NO: 71 - DNA, 65 bp, Artificial Sequence
  Example of Intenal control constructed with SEQ ID No 9, SEQ ID No 19, and reverse complementary of SEQ ID No 14 (i.e. SEQ ID No 42)
tttaggttcggcacctcttgacgtagtggcctcggcgccagtagctgctaaaagtcggtctcgcc

SEQ ID NO: 72 - DNA, 66 bp, Artificial Sequence
  Example of Intenal control constructed with SEQ ID No 9, SEQ ID No 19, and reverse complementary of SEQ ID No 16 (i.e. SEQ ID No 43)
tttaggttcggcacctcttgacgtagtggcctcggcgccagtagctgatgaaacgcgtgggtaaac

SEQ ID NO: 73 - DNA, 66 bp, Artificial Sequence
  Example of Intenal control constructed with SEQ ID No 9, SEQ ID No 19, and reverse complementary of SEQ ID No 18 (i.e. SEQ ID No 44)
tttaggttcggcacctcttgacgtagtggcctcggcgccagtagctctgttgatttataccggccg

SEQ ID NO: 74 - DNA, 67 bp, Artificial Sequence
  Example of Intenal control constructed with SEQ ID No11, SEQ ID No 19, and reverse complementary of SEQ ID No 2 (i.e. SEQ ID No 36)
caacatgaccgatgacaaggacgtagtggcctcggcgccagtagctcaaatcatcgacggtaacctc

SEQ ID NO: 75 - DNA, 66 bp, Artificial Sequence
  Example of Intenal control constructed with SEQ ID No 11, SEQ ID No 19, and reverse complementary of SEQ ID No 4 (i.e. SEQ ID No 37)
caacatgaccgatgacaaggacgtagtggcctcggcgccagtagctctgcaagtcctaagacgcca

SEQ ID NO: 76 - DNA, 70 bp, Artificial Sequence
  Example of Intenal control constructed with SEQ ID No 11, SEQ ID No 19, and reverse complementary of SEQ ID No 6 (i.e. SEQ ID No 38)
caacatgaccgatgacaaggacgtagtggcctcggcgccagtagctgccaatcgaaaagaaacacgcgga

SEQ ID NO: 77 - DNA, 65 bp, Artificial Sequence
  Example of Intenal control constructed with SEQ ID No 11, SEQ ID No 19, and reverse complementary of SEQ ID No 14 (i.e. SEQ ID No 42)
caacatgaccgatgacaaggacgtagtggcctcggcgccagtagctgctaaaagtcggtctcgcc

SEQ ID NO: 78 - DNA, 66 bp, Artificial Sequence
  Example of Intenal control constructed with SEQ ID No 11, SEQ ID No 19, and reverse complementary of SEQ ID No 16 (i.e. SEQ ID No 43)
caacatgaccgatgacaaggacgtagtggcctcggcgccagtagctgatgaaacgcgtgggtaaac

SEQ ID NO: 79 - DNA, 66 bp, Artificial Sequence
  Example of Intenal control constructed with SEQ ID No 11, SEQ ID No 19, and reverse complementary of SEQ ID No 18 (i.e. SEQ ID No 44)
caacatgaccgatgacaaggacgtagtggcctcggcgccagtagctctgttgatttataccggccg

SEQ ID NO: 80 - DNA, 68 bp, Artificial Sequence
  Example of Intenal control constructed with SEQ ID No13, SEQ ID No 19, and reverse complementary of SEQ ID No 2 (i.e. SEQ ID No 36)
cggactcaccaggagattacaacgtagtggcctcggcgccagtagctcaaatcatcgacggtaacctc

SEQ ID NO: 81 - DNA, 67 bp, Artificial Sequence
  Example of Intenal control constructed with SEQ ID No 13, SEQ ID No 19, and reverse complementary of SEQ ID No 4 (i.e. SEQ ID No 37)
cggactcaccaggagattacaacgtagtggcctcggcgccagtagctctgcaagtcctaagacgcca

SEQ ID NO: 82 - DNA, 71 bp, Artificial Sequence
  Example of Intenal control constructed with SEQ ID No 13, SEQ ID No 19, and reverse complementary of SEQ ID No 6 (i.e. SEQ ID No 38)
cggactcaccaggagattacaacgtagtggcctcggcgccagtagctgccaatcgaaaagaaacacgcgga

SEQ ID NO: 83 - DNA, 67 bp, Artificial Sequence
  Example of Intenal control constructed with SEQ ID No 13, SEQ ID No 19, and reverse complementary of SEQ ID No 10 (i.e. SEQ ID No 40)
cggactcaccaggagattacaacgtagtggcctcggcgccagtagctcaacatgaccgatgacaagg

SEQ ID NO: 84 - DNA, 67 bp, Artificial Sequence
  Example of Intenal control constructed with SEQ ID No 13, SEQ ID No 19, and reverse complementary of SEQ ID No 12 (i.e. SEQ ID No 41)
cggactcaccaggagattacaacgtagtggcctcggcgccagtagctgattacgcgaaagataccgc

SEQ ID NO: 85 - DNA, 67 bp, Artificial Sequence
  Example of Intenal control constructed with SEQ ID No15, SEQ ID No 19, and reverse complementary of SEQ ID No 2 (i.e. SEQ ID No 36)
cttttagcgaagccatggagacgtagtggcctcggcgccagtagctcaaatcatcgacggtaacctc

SEQ ID NO: 86 - DNA, 66 bp, Artificial Sequence
  Example of Intenal control constructed with SEQ ID No 15, SEQ ID No 19, and reverse complementary of SEQ ID No 4 (i.e. SEQ ID No 37)
cttttagcgaagccatggagacgtagtggcctcggcgccagtagctctgcaagtcctaagacgcca

SEQ ID NO: 87 - DNA, 70 bp, Artificial Sequence
  Example of Intenal control constructed with SEQ ID No 15, SEQ ID No 19, and reverse complementary of SEQ ID No 6 (i.e. SEQ ID No 38)
cttttagcgaagccatggagacgtagtggcctcggcgccagtagctgccaatcgaaaagaaacacgcgga

SEQ ID NO: 88 - DNA, 69 bp, Artificial Sequence
  Example of Intenal control constructed with SEQ ID No 15, SEQ ID No 19, and reverse complementary of SEQ ID No 8 (i.e. SEQ ID No 39)
cttttagcgaagccatggagacgtagtggcctcggcgccagtagcttggtacgggtaatgaaaatgatc

SEQ ID NO: 89 - DNA, 66 bp, Artificial Sequence
  Example of Intenal control constructed with SEQ ID No 15, SEQ ID No 19, and reverse complementary of SEQ ID No 10 (i.e. SEQ ID No 40)
cttttagcgaagccatggagacgtagtggcctcggcgccagtagctcaacatgaccgatgacaagg

SEQ ID NO: 90 - DNA, 66 bp, Artificial Sequence
  Example of Intenal control constructed with SEQ ID No 15, SEQ ID No 19, and reverse complementary of SEQ ID No 12 (i.e. SEQ ID No 41)
cttttagcgaagccatggagacgtagtggcctcggcgccagtagctgattacgcgaaagataccgc

SEQ ID NO: 91 - DNA, 67 bp, Artificial Sequence
  Example of Intenal control constructed with SEQ ID No 17, SEQ ID No 19, and reverse complementary of SEQ ID No 2 (i.e. SEQ ID No 36)
cgctgtttaccggatttctcacgtagtggcctcggcgccagtagctcaaatcatcgacggtaacctc

SEQ ID NO: 92 - DNA, 66 bp, Artificial Sequence
  Example of Intenal control constructed with SEQ ID No 17, SEQ ID No 19, and reverse complementary of SEQ ID No 4 (i.e. SEQ ID No 37)
cgctgtttaccggatttctcacgtagtggcctcggcgccagtagctctgcaagtcctaagacgcca

SEQ ID NO: 93 - DNA, 70 bp, Artificial Sequence
  Example of Intenal control constructed with SEQ ID No 17, SEQ ID No 19, and reverse complementary of SEQ ID No 6 o(i.e. SEQ ID No 38)
cgctgtttaccggatttctcacgtagtggcctcggcgccagtagctgccaatcgaaaagaaacacgcgga

SEQ ID NO: 94 - DNA, 69 bp, Artificial Sequence
  Example of Intenal control constructed with SEQ ID No 17, SEQ ID No 19, and reverse complementary of SEQ ID No 8 (i.e. SEQ ID No 39)
cgctgtttaccggatttctcacgtagtggcctcggcgccagtagcttggtacgggtaatgaaaatgatc

SEQ ID NO: 95 - DNA, 66 bp, Artificial Sequence
  Example of Intenal control constructed with SEQ ID No 17, SEQ ID No 19, and reverse complementary of SEQ ID No 10 (i.e. SEQ ID No 40)
cgctgtttaccggatttctcacgtagtggcctcggcgccagtagctcaacatgaccgatgacaagg

SEQ ID NO: 96 - DNA, 66 bp, Artificial Sequence
  Example of Intenal control constructed with SEQ ID No 17, SEQ ID No 19, and reverse complementary of SEQ ID No 12 (i.e. SEQ ID No 41)
cgctgtttaccggatttctcacgtagtggcctcggcgccagtagctgattacgcgaaagataccgc